(19) United States
(12) Patent Application Publication
(10) Pub. No.: US 2005/0214290 A1
(43) Pub. Date: Sep. 29, 2005

(54) Methods of blocking tissue destruction by autoreactive T cells

(76) Inventors: Yang Liu, Columbus, OH (US); Pan Zheng, Columbus, OH (US); Xuefeng Bai, Columbus, OH (US); Xingluo Liu, Powell, OH (US)

Correspondence Address: CALFEE HALTER & GRISWOLD, LLP, 800 SUPERIOR AVENUE, SUITE 1400, CLEVELAND, OH, 44114, US

(21) Appl. No.: 11/128,709

(22) Filed: May 13, 2005

Related U.S. Application Data
(62) Division of application No. 10/119,637, filed on Apr. 10, 2002.
Application No. 10/119,637 is a continuation-in-part of application No. 09/822,851, filed on Mar. 29, 2001, now abandoned.

(51) Int. Cl. A61K 39/395; A61K 38/17

(52) U.S. Cl. 424/144.1; 514/12; 514/8

(57) ABSTRACT

Methods for blocking autoreactive T cell-initiated destruction of tissues in a mammal are provided. In one embodiment, the methods involve administering a pharmaceutical composition comprising an agent selected from a fusion protein comprising an HSA/CD24 polypeptide and a fusion protein comprising a biologically active fragment of an HSA/CD24 polypeptide. In some embodiments, the polypeptide or fragment is glycosylated.

### REFERENCE TO RELATED APPLICATION

This application is a continuation-in part of U.S. application Ser. No. 09/822,851 which was filed on Mar. 29, 2001, and claims priority from U.S. Provisional Patent Application No. 60/192,814, filed on Mar. 29, 2000.

### STATEMENT OF GOVERNMENT SUPPORT

This invention is supported, at least in part, by Grant No. AI32981 from the National Institute of Health, USA. The U.S. government has certain rights in this invention.

### BACKGROUND OF THE INVENTION

The present invention relates to agents and methods for blocking deleterious T cell mediated immune reactions. Such reactions occur in autoimmune diseases, such as for example, multiple sclerosis (MS), rheumatoid arthritis, systemic lupus erythematosis, psoriasis, diabetes, and allergies. Such reactions also occur during rejection of transplants.

In theory, autoimmune diseases can be prevented by blocking activation of T cells and formation of autoreactive T cells. Accordingly, there are a number of studies being conducted to identify methods or agents that can be used to block activation of T cells (J Clin Invest. 1995 June; 95(6):2783-9; J Med. Chem. 2002 Jan. 17; 45(2):275-83). Unfortunately, since patients with autoimmune diseases have already developed autoreactive T cells, these methods have limited value for treatment of autoimmune diseases. Moreover, agents that prevent systemic T cell activation often cause serious side effects. For example, treatment with agents that block activation of T cells can also render the patient more susceptible to infections and cancer. Thus, it is desirable to have new methods for treating autoimmune diseases. A method which reduces the destruction of targeted tissues that is initiated by autoreactive T cells is especially desirable.

### SUMMARY OF THE INVENTION

The present invention provides methods for blocking or reducing autoreactive T cell-initiated destruction of tissues in a mammal. The methods employ an agent that inhibits or reduces interaction of the CD24 polypeptide with its functional ligand. The CD24 polypeptide is found on the cell membrane of activated T cells and other cell types, such as B cells, dendritic cells, epithelial cells and vascular endothelial cells.

In one embodiment, the method comprises administering a pharmaceutical composition comprising a biologically effective amount of an isolated and purified polypeptide, referred to hereinafter as the “HSA/CD24” polypeptide, a fusion protein comprising the HSA/CD24 polypeptide polypeptide, or a biologically active fragment of the HSA/CD24 polypeptide to a mammal in need of the same, i.e., a mammal who is suspected of having, known to have, or predisposed to have an autoimmune disease. As used herein, “mammal” refers to rats, mice, cats, dogs, cows, pigs, rabbits, and primates. Exemplary primates include monkeys, chimpanzees, and humans. As used herein the term “HSA/CD24” refers not only to the protein portion of the heat stable antigen (HSA) found on the surface of mouse cells but also to the mammalian homologs of mouse HSA. Thus, the term “HSA/CD24”, as used in the present application, encompasses the polypeptide portion of human CD24 and rat CD24, the known human and rat homologs of mouse HSA. Preferably, the HSA/CD24 polypeptide is glycosylated. The fusion protein comprises the HSA/CD24 polypeptide or a truncated form of the HSA/CD24 linked by a peptide bond to a peptide or protein tag. In a preferred embodiment, the HSA/CD24 fragment comprises the core region of the HSA/CD24 polypeptide.

In another embodiment, the method comprises administering a pharmaceutical composition comprising a biologically effective amount of an anti-HSA/CD24 antibody or anti-HSA/CD24 Fab fragments to a mammal known to have, suspected of having, or predisposed to having an autoimmune disease.

In another aspect, the method comprises administering to the subject an agent that reduces expression of the CD24 polypeptide in T cells. Such methods employ agents that disrupt the function of the CD24 gene at the genomic, transcriptional, post-transcriptional and translational levels. In one embodiment, the agent is an antisense molecule (referred to hereinafter as “CD24 antisense”) which reduces transcription of the CD24 gene or translation of the CD24 gene transcript in autoreactive T cells. In another embodiment, the agent is a double stranded RNA molecule (referred to hereinafter as “CD24 dsRNAi”) which interferes with expression of the CD24 gene.

The present invention also relates to a method of blocking binding of autoreactive T cells to vascular endothelial cells. In one aspect, the method comprises contacting the vascular endothelial cells with a sufficient amount of an HSA/CD24 polypeptide or a fragment thereof, or a fusion protein comprising HSA/CD24 polypeptide or a fragment thereof, or anti-HSA antibodies to inhibit interaction of the autoreactive T cells with the vascular endothelial cells. In another aspect, the method comprises introducing an oligonucleotide or polynucleotide that inhibits expression of the CD24 polypeptide into the autoreactive T cell, or the vascular endothelial cell or both. Examples of such oligonucleotides and polynucleotides include, but are not limited to, a CD24 antisense oligonucleotide, an expression vector comprising a polynucleotide or nucleic acid encoding a CD24 antisense oligonucleotide, a CD24 dsRNAi, and an expression vector comprising a polynucleotide or nucleic acid encoding a CD24 dsRNAi.

The present invention also relates to isolated and purified HSA/CD24 fusion proteins employed in the above-described methods and to transgenic or knock in mice that express the human CD24 protein on their T cells or their vascular endothelial cells or all other cell types that normally express CD24 but, as a result of targeted mutation, do not express murine HSA on any cells. Such mice provide a unique model to test the effectiveness of drugs designed to block or enhance the biological function of human CD24-mediated autoimmune diseases.

### BRIEF DESCRIPTION OF THE FIGURES

FIG. 1. Targeted mutations of HSA and CD28 reveal two distinct checkpoints in the development of EAE. a. Targeted mutations of either HSA or CD28 prevent induction of EAE. WT, CD28(−/−) or HSA(−/−) mice were immunized with MOG peptide. Clinical signs were scored as described in the method section. b. Proliferative response of lymph node T cells to MOG peptides. Draining lymph node cells from day 10-immunized mice were stimulated with given concentrations of MOG peptide and irradiated syngeneic naive spleen cells as antigen-presenting cells. c. Enumeration of cytokine-producers by ELISpot. Draining lymph node cells used in b were used as responder cells. The numbers of cells secreting either IL2, ILA, and IFNγ among 1×10⁶ lymph node cells in response to MOG peptide (AA35-55) were presented. Data shown were means+/−SEM from three independent experiments.

FIG. 2. Histological analysis of spinal cord of MOG immunized WT or HSA(−/−) mice. a: The means and SEM of histological scores of WT and HSA(−/−) mice spinal cords. Ten independent cross sections, from cervical to sacral regions, were examined in each spinal cord. The data are summarized from 30 spinal cord sections from 3 mice in each group. b. Representative histology in immunized WT mice, all sections examined contain histology lesions. c and d. Histology sections (100×) of immunized HSA(−/−) mice. A lesion-free section is presented in c, while a lesion containing section is presented in d.

FIG. 3. Requirement for HSA expression on both T cells and non-T host cells for the induction of EAE. Histology (63× for a, b, c and the left panel of d; 200× for the right panel of d) of spinal cords of the HSA(−/−)(a, b) or WT(c, d) recipient mice on day 12 after adoptive transfer. Draining lymph node cells were isolated from either WT or HSA(/−) mice after immunization, and were stimulated with antigen and IL2 for 4 days in vitro. The activated T cells were injected into either WT or HSA(−/−) mice (100×10⁶ cells per mouse). EAE development was monitored daily for clinical signs. At 12 days after transfer, recipient mice were sacrificed and spinal cords were processed for histological examination. No disease was observed in WT>HSA(−/−), HSA(−/−)>WT, or HSA(−/)>HSA(−/−) recipients.

FIG. 4. Clinical scores of the adoptive transfer experiment with 4 (WT>HSA(−/−) and HSA(−/−)>WT groups) or 5 (WT>WT and HSA(−/−)>HSA(−/−) groups) mice per group.

FIG. 5. Transgenic expression of HSA exclusively on T cell lineage is insufficient for EAE development. a. Phenotypes of WT, HSA-TG, HSA(−/−), and HSATG/HSA(−/−) mice by flow cytometry using anti-HSA and anti-CD3 mAbs. b. EAE score in WT, HSATG, HSA(−/−), and HSATG/HSA(−/−) mice after immunization with the MOG peptides.

FIG. 6. HSAIg ameliorates EAE. a. Analysis of HSAIg by SDS-PAGE. 10 μg of purified HSAIg was separated by 10% reducing (R) and non-reducing SDS-PAGE. The proteins were stained by Comassie blue. The EAE score for control (PBS) or HSAIg-treated mice. EAE was induced in WT mice as described in Materials and Methods. On days 8, 10, 12, 14 and 22 after immunization, five mice per group were injected (i.p.) with 100 μg/mouse of either HSAIg or 100 ml of PBS as control. The effect of HSAIg has been evaluated in three independent experiments with similar results.

FIG. 7 shows the amino acid sequence, SEQ ID NO. 1, of the mouse HSA polypeptide. The signal peptide extends from amino acid 1 through amino acid 26 of the sequence. The glycophosphatidyl (GPI) anchor region includes and extends from amino acid 54 through amino acid 76.

FIG. 8 shows the amino acid sequence, SEQ ID NO. 2, of the human CD24 polypeptide. The signal peptide extends from amino acid 1 through amino acid 26 of the sequence. The glycophosphatidyl (GPI) anchor region includes and extends from amino acid 60 through amino acid 80.

FIG. 9 shows the amino acid sequence, SEQ ID NO. 3, of the rat CD24 polypeptide. The signal peptide extends from amino acid 1 through amino acid 26 of the sequence. The glycophosphatidyl (GPI) anchor region includes and extends from amino acid 57 through amino acid 76.

FIG. 10 shows the DNA sequence, SEQ ID NO. 4, of a fusion gene which comprises a nucleotide sequence encoding HSA fused to the genomic sequence of human IgG1 Fc. The predicted sequence of the cDNA, SEQ ID NO. 5, which results from splicing of the introns IgG1 Fc sequence and the predicted amino acid sequence, SEQ ID NO. 14, are also shown in this figure. The normal font with under line is HSA sequence, bold phase is new sequence, italics is IgG1 Fc sequence.

FIG. 11 is a diagram of a construct for producing human CD24 gene knock-in mice. Arm 1 of the knock in construct comprises nucleotide 2001 through nucleotide 5500 of the mouse HSA/CD14 gene, GenBank Accession No. X72910. Arm 2 of the construct is a chimera gene consisting of the last 256 bp sequence of exon 1 of the mouse CD24 gene, the first 240 bp sequence of exon 2 of the human CD24 gene and about 3 kb of mouse CD24 sequence which comprises remaining exon 2 sequence encoding for 3′ untranslated region and 3′ sequence of the mouse CD24 gene (Seq I.D. 22).

FIG. 12 is a diagram of a plasmid for producing transgenic mice expressing human CD24 in T cells. To produce this plasmid human CD24 coding sequence is subcloned into transgenic construct vector p1017Bam HI site, which is described in EMBO J. 9: 3821-3829, 1990) CD24 forward primer (CD24F.Bam): G GCC GGA TCC ATG GGC AGA GCA ATG GTG with BamH1 site 5′ to ATG start codon. CD24 reverse primer (CD24R. XhoBam): G GCC GGA TCC CTC GAG TTA AGA GTA GAG ATG CAG with Bam HI and Xho I sites 3′ to TAA stop codon.

FIG. 13 is a diagram of a vector for producing mice that express CD24 in vascular endothelial cells.

FIG. 14 shows a comparison of mouse and human CD24 cDNA sequences, and the preferred sequences to be targeted by CD24 antisense RNA and CD24 dsRNAi. Human CD24 and mouse CD24 cDNA sequences (Human CD24 (XM_{—}099027) and Mouse CD24 (NM_{—}009846)) are aligned by double blast search. The regions with a stretch of identity that are 17 or more base pairs in length are highlighted as preferred targets for CD24 antisense and dsRNAi agents.

FIG. 15 is a dot plot demonstrating inhibition of CD24 expression in CHO cells using dsRNAi. Chinese hamster ovary (CHO) cells were transiently transfected with human CD24 plasmid alone (top), the CD24 plasmid plus dsRNAi (middle) or CD24 plasmid plus inverted dsRNA as control (lower panel). The expression of CD24 on cell surface was analyzed at 72 hours after transfection using phycoerythorin-conjugated anti-human CD24 mAb (BD Pharmingen). Note essential absence of CD24-expression in cells treated with CD24 dsRNAi.

### DETAILED DESCRIPTION OF THE INVENTION

The present invention provides methods for blocking destruction of tissue by autoreactive T cells in a mammalian subject. The methods employ agents which inhibit or reduce, either directly or indirectly, the interaction of the CD24 polypeptide with its functional ligand. The CD 24 polypeptide is present on the cell membrane of activated T cells and other cell types such as B cells, dendritic cells, epithelial cells and vascular endothelial cells.

In one embodiment, the method comprises administering a pharmaceutical composition comprising a biologically effective amount of an isolated and purified HSA/CD24 polypeptide or a fragment thereof to a mammal suspected of having an autoimmune disease. In another embodiment a fusion protein comprising the HSA/CD24 polypeptide or fragment thereof linked by a peptide bond to a peptide or protein tag is administered to the mammal. Preferably, the HSA/CD24 polypeptide is glycosylated. In another embodiment an antibody which is immunospecific for the HSA/CD24 polypeptide is administered to the mammal.

In another aspect, the method comprises administering to the subject an agent that reduces expression of the CD24 polypeptide in T cells. Such methods employ agents that disrupt the function of the CD24 gene at the genomic, transcriptional, post-transcriptional and translational levels. In this aspect, embodiments of the present method employ antisense molecules or dsRNAi to inhibit expression of the CD24/HSA gene and production of the CD24 polypeptide in the autoreactive T cells of the mammalian subject The present invention also relates to a method of treating a human subject known to have, suspected of having, or predisposed to having an autoimmune disease. The methods involve treating the human subject with an agent which inhibit or reduce, either directly or indirectly, the interaction of the CD24 polypeptide which is present on the cell surface of the subject's cells, including but not limited to activated T cells, with the functional ligand of CD24. In accordance with the present invention, it is believed that such ligands include, but are not limited to, CD24 itself, P-selectin, and very late antigen 4.

In one aspect, the therapeutic method comprises administering a pharmaceutical composition comprising a biologically effective amount of an isolated and purified human CD24 polypeptide or fragment thereof, or a fusion protein comprising such molecule, to the human subject. In another embodiment of this therapeutic method, the pharmaceutical composition comprises anti-human CD24 antibodies or their Fab fragments. Preferably, the anti-human CD24 antibody is a monoclonal antibody, more preferably a humanized monoclonal anti-human CD24 antibody. Preferably, the pharmaceutical composition is administered after autoreactive T cells have been detected in the human subject.

Preferably, the pharmaceutical composition is administered by injection. The present method is useful for treating subjects suspected of having autoimmune diseases such as for example, multiple sclerosis (MS), rheumatoid arthritis, and insulin-dependent diabetes mellitus. By “treating” is meant ameliorating or tempering the severity of the condition, either occurring or expected to occur in the future. In cases of autoimmune demyelinating diseases of the CNS such as for example MS, the pharmaceutical composition is administered either when patients have clinical symptoms, or when they are in temporary remission. Preferably, the protocol involves intravenous injection. In the case of rheumatoid arthritis, the pharmaceutical composition, preferably, is administered intravenously (i.v.) after the acute symptoms are relieved by other therapeutic methods. In the case of insulin dependent diabetes mellitus, the pharmaceutical composition, preferably, is administered intravenously after autoreactive T cells are detected in the peripheral blood.

Further embodiments of this therapeutic method employ antisense molecules or dsRNAi to inhibit expression of the CD24 gene and production of CD24 polypeptide in the T cells of the human subject.

Pharmaceutical Composition

The pharmaceutical composition comprises a biologically effective amount of an HSA/CD24 polypeptide or a biologically active variant thereof or alternatively a fragment of an HSA/CD24 polypeptide or a biologically active variant thereof, and preferably a relatively inert topical carrier. Many such carriers are routinely used and can be identified by reference to pharmaceutical texts.

HSA Antigen

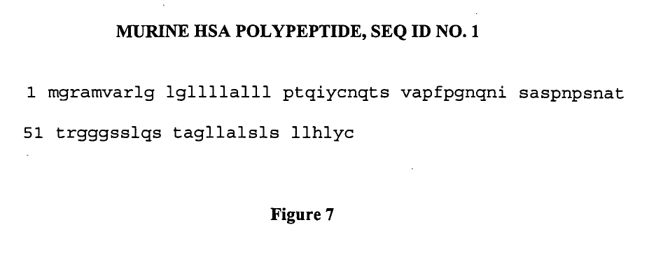

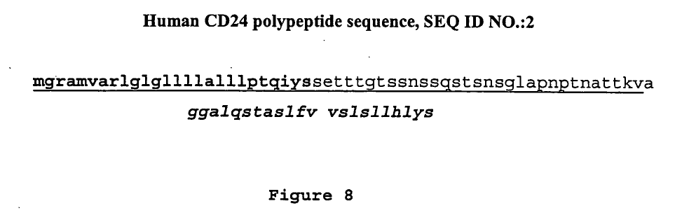

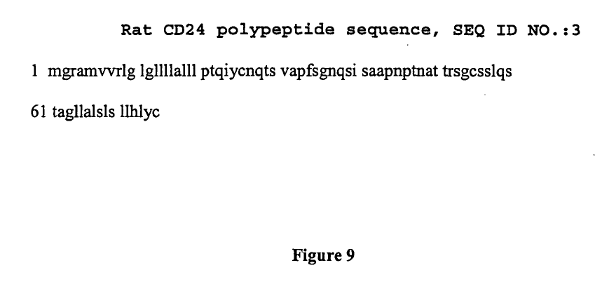

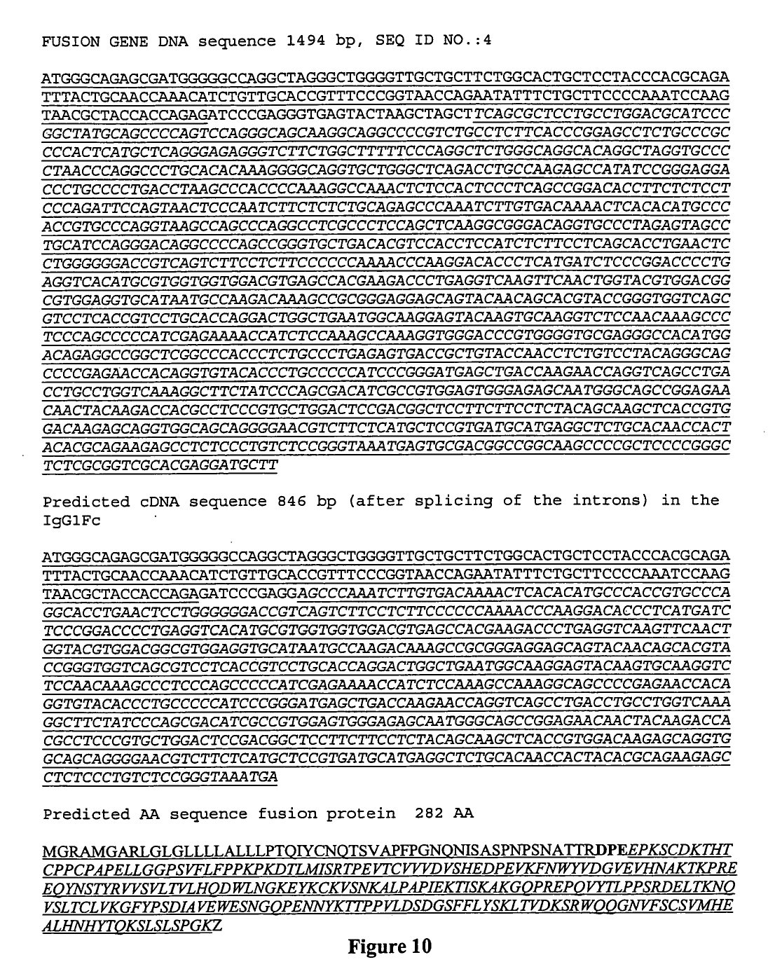

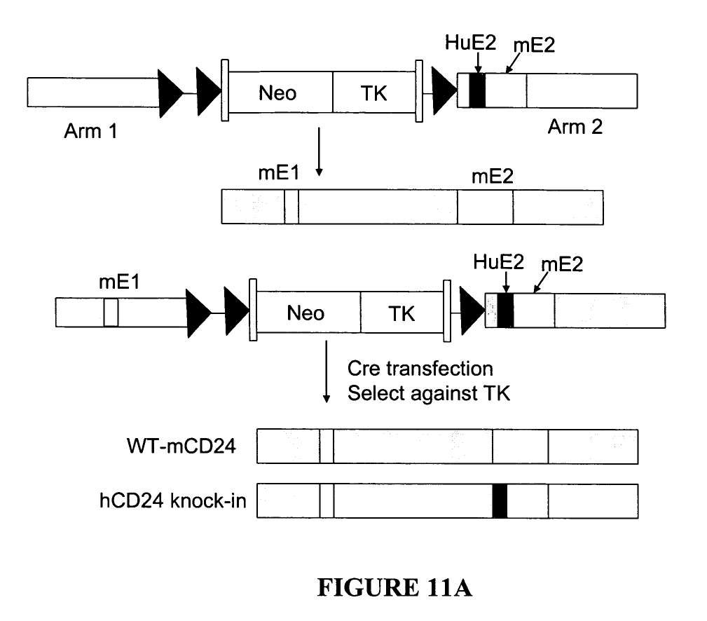

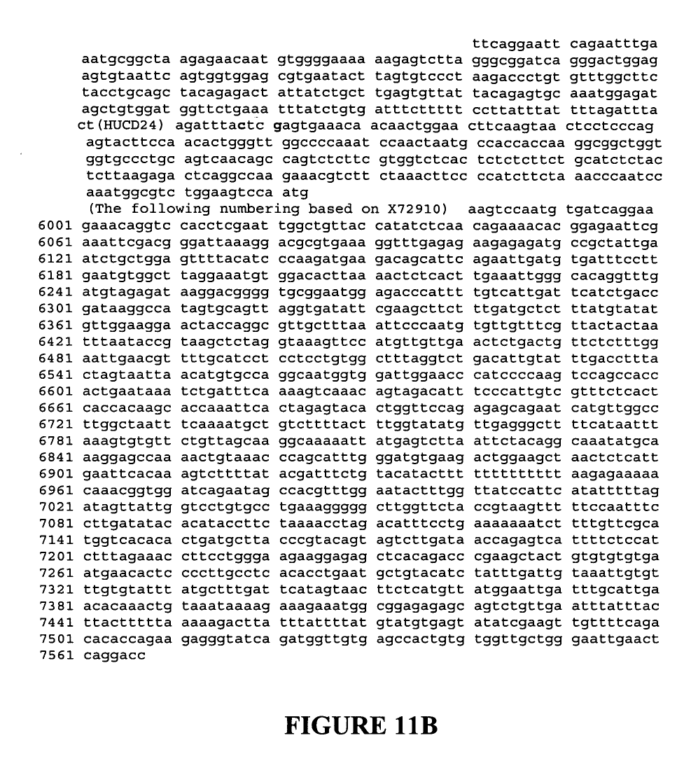

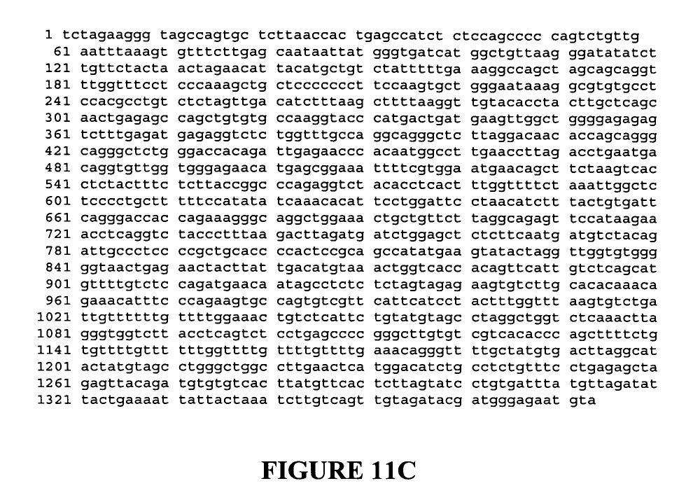

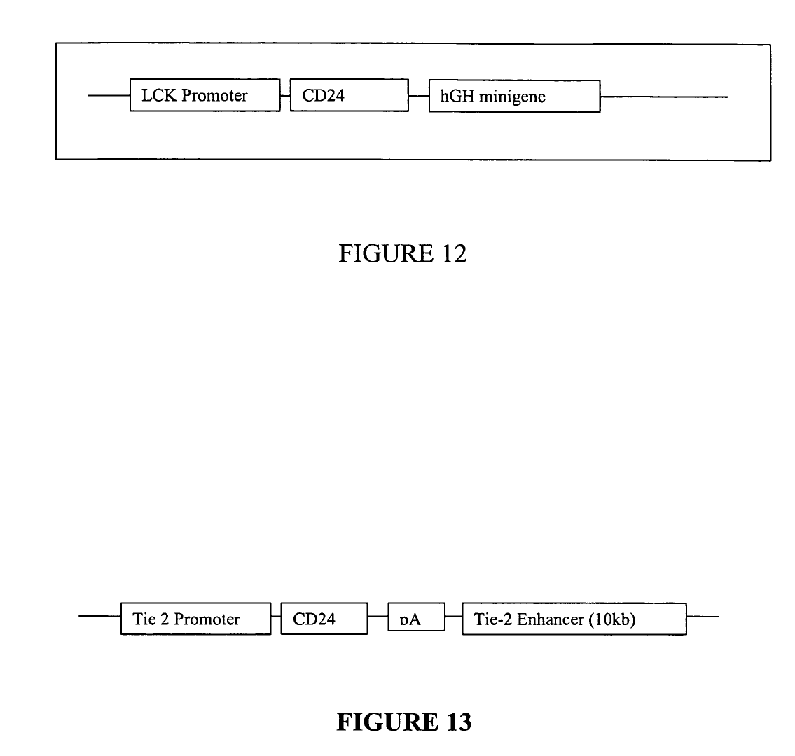

The mouse HSA antigen and the mammalian homologs thereof are polypeptides comprising approximately 76-80 amino acids. The HSA polypeptide and the mammalian homologs thereof are cell surface molecules which are linked to the cell membrane via a glycophosphatidylinositol (GPI) tail. The HSA antigen is constitutively expressed on most hematopoietic and developing neuronal cells. In some lymphocytes, such as for example T cells, expression of the HSA polypeptide is induced. As shown in FIGS. 7-9, the immature forms of mouse HSA antigen, human CD24 and rat CD24 comprise a signal sequence, a core region that is maintained in the mature protein, and a GPI anchor region. As shown in FIG. 7, the signal sequence of the mouse HSA antigen includes amino acid 1 through amino acid 26 of SEQ ID NO. 1; the core region includes amino acid 27 through amino acid 53 of SEQ ID NO. 1; and the GPI region includes amino acid 54 through amino acid 75 of SEQ ID NO. 1. As shown in FIG. 8, the signal sequence of the human CD24 antigen includes amino acid 1 through amino acid 26 of SEQ ID NO.2; the core region includes amino acid 27 through amino acid 59 of SEQ ID NO. 2 and the GPI region includes amino acid 60 through amino acid 80 of SEQ ID NO. 2. As shown in FIG. 9, the signal sequence of the rat CD24 antigen includes amino acid 1 through amino acid 26 of SEQ ID NO.3; the core region includes amino acid 27 through amino acid 56 of SEQ ID NO. 3 and the GPI region includes amino acid 57 through amino acid 76 of SEQ ID NO. 3. The nucleotide sequence of a polynucleotide which encodes the human CD4 polypeptide is available at the GenBank Accession No. AK000168. The nucleotide sequence of a cDNA which encodes the rat CD24 polypeptide is available at GenBank Accession No. AWK12164. The nucleotide sequence of a cDNA which encodes mouse HSA antigen is available at GenBank Accession M58661.

The present invention relates to novel method of using an HSA/CD24 polypeptide or fragment thereof to treat autoimmune diseases. Preferably, the polypeptide or fragment is glycosylated. In one embodiment the HSA/CD24 fragment is a truncated form of the HSA/CD24 polypeptide which lacks a few amino acids, i.e., from 1 to 2 amino acids, at the amino terminus or carboxy terminus thereof. In another embodiment the HSA/CD24 fragment is a polypeptide which comprises essentially only the core region of the HSA/CD24 polypeptide, i.e. the HSA/CD24 fragment lacks most or all of the signal peptide and most or all of the CPI anchor region. As used herein the term HSA/CD24 polypeptide comprises all mammalian homologs of mouse HSA, including human CD24 and rat CD24.

The HSA/CD24 polypeptide or HSA/CD24 fragment that is used in the pharmaceutical composition is the naturally-occurring HSA/CD24 polypeptide, a biologically active fragment of the naturally-occurring HSA/CD24 polypeptide, a biologically active variant of the naturally-occurring HSA/CD24 polypeptide, or a biologically active variant of a fragment of the naturally-occurring HSA/CD24 polypeptide The biologically active variant of the HSA/CD24 polypeptide has an amino acid sequence which is at least 80%, more preferably at least 93%, most preferably at least 96% identical to the amino acid sequence of the naturally occurring HSA/CD24 polypeptide that is present in the mammal to whom the pharmaceutical composition is being administered. Similarly, the biologically active variant of the fragment of the HSA/CD24 polypeptide has an amino acid sequence which is at least 80%, preferably at least 90%, more preferably at least 95% identical to the amino acid sequence of the corresponding naturally-occurring HSA/CD24 fragment. For murine HSA, alteration in Positions 1(Asn), 4(Ser), 13(Asn), 15(Ser), 17(Ser), 21(Ser), 22(Asn), 24(Thr) and 25(Thr) of the mature peptide, i.e., the peptide which lacks the signal peptide, may alter glycosylation and interfere with its ability to block destruction of tissue by autoreactive T cells. Thus, it is preferred that alternations not be made at these sites. In the human homologue of HSA, i.e., human CD24, 20 out of 31 amino acids are potential glycosylation sites.

An HSA/CD24 polypeptide which is less than 100% identical to the naturally occurring HSA/CD24 polypeptide has an altered sequence in which one or more of the amino acids in the HSA homologue is deleted or substituted, or one or more amino acids are inserted into the sequence of the naturally occurring HSA/CD24 polypeptide. HSA/CD24 sequences which are at least 95% identical to the naturally occurring HSA/CD24 sequence have no more than 5 alterations, i.e., any combination of deletions, insertions or substitutions, per 100 amino acids of the reference sequence. Percent identity is determined by comparing the amino acid sequence of the altered HSA/CD24 sequence with the naturally occurring sequence using MEGALIGN project in the DNA STAR program. Sequences are aligned for identity calculations using the method of the software basic local alignment search tool in the BLAST network service (the National Center for Biotechnology Information, Bethesda, Md.) which employs the method of Altschul, S. F., Gish, W., Miller, W., Myers, E. W. & Lipman, D. J. (1990) J. Mol. Biol. 215, 403-410. Identities are calculated by the Align program (DNAstar, Inc.) In all cases, internal gaps and amino acid insertions in the candidate sequence as aligned are not ignored when making the identity calculation.

While it is possible to have nonconservative amino acid substitutions, it is preferred that the substitutions be conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydroxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic, residue, e.g. phenylalanine and tyrosine, with another; replacement of one basic residue, e.g. lysine, arginine and histidine, with another; and replacement of one small amino acid, e.g., alanine, serine, threonine, methionine, and glycine, with another.

The biologically active fragments and variants of a naturally-occurring HSA/CD24 have an ID₅₀ which is comparable to, i.e., not more than twice the value of, the ID₅₀ of the corresponding naturally-occurring HSA/CD24 polypeptide. The ID₅₀ of the HSA/CD24 variant or fragment and its corresponding naturally-occurring polypeptide is determined by measuring the amount of these polypeptides needed to reduce the clinical symptoms in experimental autoimmune models, such as EAE or Type II diabetes in NOD mouse or rat. Alternatively, one can determine the ID₅₀ of the biologically active HSA/CD24 variant or fragment and its corresponding naturally occurring HSA/CD24 polypeptide by an adhesion assay or assays that measure migration of T cells through endothelial cell monolayer in transwell culture. The amount of the biologically active variant of the HSA/CD24 polypeptide or fragment thereof needed to reduce binding of activated T cells to vascular endothelial cells by at least 50%, preferably, is no greater than twice the amount of the corresponding naturally occurring HSA/CD24 polypeptide or fragment thereof.

The present method also employs fusion proteins comprising an HSA/CD24 polypeptide or a biologically active fragment thereof and a tag, i.e., or one or more amino acids, preferably from about 5 to 300 amino acids which are added to the amino terminus of, the carboxy terminus of, or any point within the amino acid sequence of the HSA/CD24 polypeptide or the biologically active fragment thereof. Preferably, the HSA/CD24 polypeptide or core region thereof is glycosylated. Typically, such additions are made to simplify purification of an expressed recombinant form of the corresponding HSA/CD24 polypeptide or core region thereof. Such tags are known in the art. Representative examples of such tags include sequences which encode a series of histidine residues, the epitope tag FLAG, the Herpes simplex glycoprotein D, beta-galactosidase, maltose binding protein, or glutathione S-transferase. Preferably, the fusion protein comprises the HSA polypeptide or a fragment thereof linked by a peptide bond to the hinge-CH2—CH3 regions of human immunoglobin Gl (“IgG1”). The fusion protein can be easily purified by affinity chromatography using either anti-IgG or protein A or protein G. Since IgG is not immunogenic in humans, the fusion protein can be administrated repeatedly if necessary.

Methods of Preparing the HSA/CD24 Polypeptide or Fusion Protein

The HSA/CD24 polypeptides and fusion proteins may be produced by using cell-free translation systems and RNA molecules derived from DNA constructs that encode the polypeptide or fusion protein. Preferably, the HSA/CD24 polypeptide or fusion protein is made by transfecting host cells with expression vectors that comprise a DNA sequence that encodes the respective HSA/CD24 polypeptide or fusion protein and then inducing expression of the polypeptide in the host cells. For recombinant production, recombinant constructs comprising one or more of the sequences which encode the HSA/CD24 polypeptide or fusion protein are introduced into host cells by conventional methods such as calcium phosphate transfection, DEAE-dextran mediated transfection, transvection, microinjection, cationic lipid-mediated transfection, electroporation, transduction, scrape lading, ballistic introduction or infection.

The HSA/CD24 polypeptide or fusion protein may be expressed in suitable host cells, such as for example, mammalian cells, yeast, insect cells or other cells under the control of appropriate promoters using conventional techniques. Suitable hosts include, but are not limited to, CHO, COS cells and 293 HEK cells. Following transformation of the suitable host strain and growth of the host strain to an appropriate cell density, the cells are harvested by centrifugation, disrupted by physical or chemical means, and the resulting crude extract retained for further purification of the epitope or chimeric peptide. For obtaining properly glycosylated forms of the protein, it is preferred that CHO cells be used.

Conventional procedures for isolating recombinant proteins from transformed host cells, such as isolation by initial extraction from cell pellets or from cell culture medium, followed by salting-out, and one or more chromatography steps, including aqueous ion exchange chromatography, size exclusion chromatography steps, and high performance liquid chromatography (HPLC), and affinity chromatography may be used to isolate the recombinant polypeptide.

Carrier

The acceptable carrier is a physiologically acceptable diluent or adjuvant. The term physiologically acceptable means a non-toxic material that does not interfere with the effectiveness of HSA. The characteristics of the carrier will depend on the route of administration and particular compound or combination of compounds in the composition. Preparation of such formulations is within the level of skill in the art. The composition may further contain other agents which either enhance the activity of the HSA or complement its activity. The composition may further comprise fillers, salts, buffers, stabilizers, solubilizers, and other materials well known in the art.

Dosage

A biologically effective amount is an amount sufficient to partially or completely block destruction of the targeted tissue initiated by the autoreactive T cell or to ameliorate the pathological effects of the autoimmune disease. The effective amount can be achieved by one administration of the composition. Alternatively, the effective amount is achieved by multiple administration of the composition to the mammal.

Antibodies

The invention further provides a therapeutic method which comprises administering a pharmaceutically effective amount of an anti-HSA/CD24 antibody, preferably a humanized anti-HSA/CD24 antibody, to a human subject suspected of having an autoimmune disease. The anti-HSA/CD24 antibody is immunospecific for the HSA/CD24 polypeptide meaning the antibody has substantially greater affinity for the HSA/CD24 polypeptide than for other polypeptides that are found on the T cells of the mammal being treated. Various forms of an anti-HSA/CD24 antibody may be used in this therapeutic method. For example, the anti-HSA/CD24 antibody may be a full length antibody (e.g., having a human immunoglobulin constant region) or an antibody fragment (e.g. a F(ab′)₂).

The term “antibody” as used herein encompasses monoclonal antibodies (including full length monoclonal antibodies), polyclonal antibodies, multispecific antibodies (e.g., bispecific antibodies), and antibody fragments so long as they exhibit the desired biological activity. “Antibody fragments” comprise a portion of a full length antibody, generally the antigen binding or variable region thereof. Examples of antibody fragments include Fab, Fab′, F(ab′)₂, and Fv fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The monoclonal antibodies to be used in accordance with the present invention may be made by the hybridoma method first described by Kohler et al., Nature 256: 495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352: 624-628 (1991) and Marks et al., J. Mol. Biol. 222: 581-597 (1991), for example.

The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step

In order to avoid potential immunogenicity of the mAbs in human, the mAbs that have desired function are preferably humanized. “Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321: 522-525 (1986); Reichmann et al., Nature 332: 323-329 (1988); and Presta, Curr. Op. Struct. Biol. 2: 593-596 (1992).

Alternatively, transgenic mice with human IgV and IgC genes may be used to produce human mAb specific for human CD24. These mice are available from Abgenix, Inc., and Mederex, Inc, and the art has been described fully (Nature Genetics, 1997, 15:146).

Antisense Molecules

In certain aspects, the present therapeutic methods employ agents which reduce or inhibit expression of the CD24 gene or production of the CD24 polypeptide in the T cells of human subjects known to have or suspected of having an autoimmune disease, such as multiple sclerosis, rheumatoid arthritis, and type II diabetes.

One such agent is an antisense molecule. The antisense molecule for CD24 is an oligomer which comprises from 20 to 200 bases, preferably less than 100 bases, and is targeted to a nucleic acid encoding the human CD24 polypeptide, in other words, the human CD24 gene or mRNA expressed from the human CD24 gene. The targeting process involves determination of a site or sites within the nucleic acid sequence of the CD24 gene or mRNA for the oligonucleotide interaction to occur such that transcription of the CD24 gene or translation of the CD24 mRNA will be reduced in the human subject's T cells. Once the target site or sites have been identified, oligonucleotides are chosen which are sufficiently complementary to the target, i.e., hybridize sufficiently well and with sufficient specificity, to give the desired reduction in expression of the CD24 polypeptide. Such inhibition can be measured in ways which are routine in the art, for example by Northern blot assay of mRNA expression or Western blot assay of protein expression, or flow cytometry analysis for cell surface expression.

“Hybridization”, as used herein means hydrogen bonding, also known as Watson-Crick base pairing, between complementary bases, usually on opposite nucleic acid strands or two regions of a nucleic acid strand. “Specifically hybridizable” and “complementary” are terms which are used to indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted. Affinity of an oligonucleotide for its target (in this case a nucleic acid encoding CD24 polypeptide) is routinely determined by measuring the Tm of an oligonucleotide/target pair, which is the temperature at which the oligonucleotide and target dissociate; dissociation is detected spectrophotometrically. The higher the Tm, the greater the affinity of the oligonucleotide for the target.

In the context of this therapeutic method, the term “oligonucleotide” refers to an oligomer or polymer of nucleotide or nucleoside monomers consisting of naturally occurring bases, sugars and intersugar (backbone) linkages. The term “oligonucleotide” also includes oligomers comprising non-naturally occurring monomers, or portions thereof, which function similarly. Modifications may be on one or more bases, sugars, or backbone linkages, or combinations of these; such modifications are well known in the art. Modified or substituted oligonucleotides are often preferred over native forms because of properties such as, for example, enhanced cellular uptake and increased stability in the presence of nucleases. A number of nucleotide and nucleoside modifications have been shown to make the oligonucleotide into which they are incorporated more resistant to nuclease digestion than the native oligodeoxynucleotide. Nuclease resistance is routinely measured by incubating oligonucleotides with cellular extracts or isolated nuclease solutions and measuring the extent of intact oligonucleotide remaining over time, usually by gel electrophoresis. Oligonucleotides which have been modified to enhance their nuclease resistance survive intact for a longer time than unmodified oligonucleotides. A variety of oligonucleotide modifications have been demonstrated to enhance or confer nuclease resistance. In some cases, oligonucleotide modifications which enhance target binding affinity are also, independently, able to enhance nuclease resistance. A discussion of antisense oligonucleotides and some desirable modifications can be found in De Mesmaeker et al. Acc. Chem. Res. 1995, 28, 366-374.

Specific examples of some oligonucleotides contemplated for the present method include those containing modified backbones, for example, phosphorothioates, phosphotriesters, methyl phosphonates, short chain alkyl or cycloalkyl intersugar linkages or short chain heteroatomic or heterocyclic intersugar linkages. The oligonucleotides may be chimeric oligonucleotides. “Chimeric oligonucleotides” as used herein mean oligonucleotides which contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the RNA target) and a region that is a substrate for RNase H cleavage. In one embodiment, a chimeric oligonucleotide comprises at least one region modified to increase target binding affinity, and, usually, a region that acts as a substrate for RNAse H. RNAse H is a cellular endonuclease that cleaves the RNA strand of RNA:DNA duplexes; activation of this enzyme therefore results in cleavage of the RNA target, and thus can greatly enhance the efficiency of antisense inhibition. Cleavage of the RNA target can be routinely demonstrated by gel electrophoresis.

Alternatively, an expression vector comprising a polynucleotide or nucleic acid encoding an antisense oligonucleotide targeted to nucleic acids that encode the CD24 polypeptide are introduced into the subject's T cells. The CD24 antisense encoding nucleic acid is operatively linked to a promoter. A “promoter” is a sequence that directs the binding of RNA polymerase and thereby promotes RNA synthesis, which in the present method is synthesis of the antisense oligonucleotide. Operatively linked is understood to mean that the CD24 antisense encoding sequence is joined to the promoter region such that the promoter is oriented 5′ to the CD24 antisense encoding sequence and is of an appropriate distance from the transcription start site, so that the transcription of the polynucleotide which encodes the CD24 antisense oligonucleotide will be dependent on or controlled by the promoter sequence. The arts of restriction enzyme digestion and nucleic acid ligation to be used in construction of the CD24 antisense encoding polynucleotide-promoter construct are well known in the art as exemplified by Maniatis et al., Molecular Cloning, A Laboratory Manual, Cold Spring Harbor, N.Y., 1982, (incorporated herein by reference). Many examples of constitutive promoters have been described in the art such as those isolated from cytomegalovirus early gene, murine MHC class I, actin, etc. An example of T-cell specific promoter is the lck promoter (EMBO J. 9: 3821-3829, 1990), which we have shown to be able to drive T cell-specific expression (Eur. J. Immunol 27:2524-2528, 1997). An example of vascular cell specific vector is described by Sato et al. (Proc. Natl. Acad Sci USA, 94:3058-63(1997)

dsRNAi

Another agent for reducing or inhibiting expression of the CD24 gene and production of the polypeptide in the T cells of human subjects is a double stranded oligonucleotide or polynucleotide known as dsRNAi. One strand of the dsRNA comprises a CD24 sense sequence; while the other strand comprises a CD24 antisense sequence. Preferably, the dsRNAi further comprises a linker connecting the antisense sequence to the sense sequence. Preferably, the CD24 sense and anti-sense sequences are from 19-30 bases in length. The linker is at least 10 bases in length, and preferably, from 10-20 bases in length. The CD24 dsRNAi prevents accumulation of CD24 mRNA in the transformed cells, most likely through a post-transcription gene silencing method known in the art as double-stranded RNA interferences.

dsRNAi can be synthesized using standard techniques. For example single-stranded RNA corresponding to the sense CD24 sequence, and single stranded RNA corresponding to the antisense CD24 sequence can be synthesized according to methods known in the art. The single stranded RNAs can then be annealed in vitro by methods known in the art, to produce the dsRNA. To increase the stability of the dsRNAi, several nucleotide with de-oxyl-nucleotide can be incorporated at the 3′ of the oligonucleotides.

Alternatively, an expression vector comprising a polynucleotide or nucleic acid encoding CD24 dsRNAi is introduced into the subject's T cells or, the subject's vascular endothelial cells, or both. Such polynucleotide comprises a sequence which encodes a sense CD24 RNA coding sequence and an antisense CD24 RNA coding sequence and a linker sequence which links the sense CD24 RNA coding sequence to the antisense CD24 RNA coding sequence. The expression vector further comprises a promoter operatively linked to the CD24 dsRNAi coding sequence.

Targeting of the CD24 Antisense and dsRNAi Molecules

In accordance with the present method, targeted regions of the CD24 gene and CD24 mRNA include not only the coding region for the CD24 polypeptide, but also the 5′-untranslated region, the 3′-untranslated region, the 5′ cap region, intron regions and intron/exon or splice junction regions of the targeted nucleic acid. The functions of messenger RNA to be interfered with include all vital functions such as translocation of the RNA to the site for protein translation, actual translation of protein from the RNA, splicing or maturation of the RNA and possibly even independent catalytic activity which may be engaged in by the RNA. The overall effect of such interference with the CD24 mRNA function is to cause interference with expression of the CD24 polypeptide.

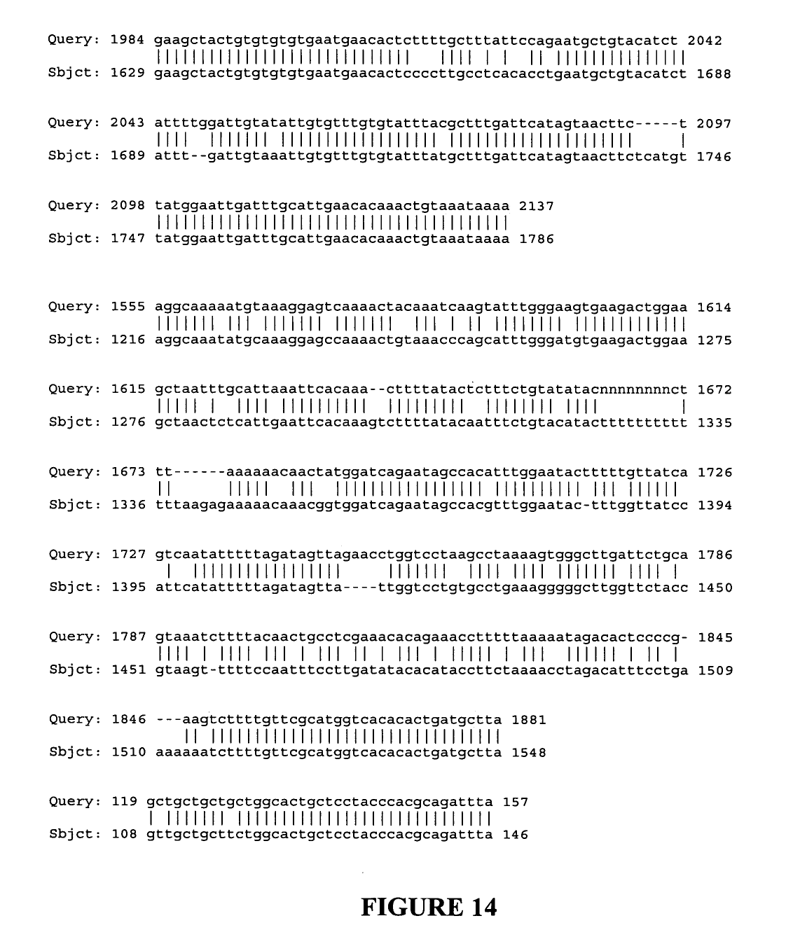

Although numerous areas can be targeted for anti-sense and dsRNAi molecules, there is a significant advantage to target areas in which the sequence is completely conserved between CD24 in mouse and man. In this way, the CD24 anti-sense and dsRNAi molecules can be screened for both efficacy and toxicity in preclinical models before they are used for human clinical trials. A comparison between human and mouse CD24 cDNA sequence, as listed in FIG. 14, revealed 8 areas within the 2.1 kb areas of human CD24 that can be as targets for antisense and dsRNAi molecules.

Delivery of the CD24 Antisense and dsRNAi Molecules

Single-stranded CD24 anti-sense oligonucleotides and dsRNAi molecules can be introduced into the subject's cells, including but not limited to T cells, vascular endothelial cells, or both. The molecules are introduced into the cells either ex vivo or in vivo. “Ex vivo” means that these molecules are introduced into the T cells or endothelial cells outside the body of the subject from whom the T cells or endothelial cells are obtained. The cells are then re-introduced back into the subject. For in vivo delivery to these target cells, the CD24 antisense and dsRNAi molecules are introduced into the subject by injection. Preferably, the injection is intravenous, or intralesional injection (as in the case of rheumatoid arthritis).

Delivery of the CD24 antisense or CD24 dsRNAi encoding polynucleotide-promoter construct into the subject may be either direct, in which case the subject is directly exposed to the construct or construct-carrying vector, or indirect, in which case cells are first transformed with the construct in vitro, then transplanted into the patient. The latter method is referred to as cell-based gene-therapy.

A retroviral vector may be used to deliver the CD24 antisense and CD24 dsRNAi encoding construct (see Miller et al., 1993, Meth. Enzymol. 217:581-599). Retroviral vectors have been modified to delete retroviral sequences that are not necessary for packaging of the viral genome and are maintained in infected cells by integration into genomic sites upon cell division. More detail about retroviral vectors can be found in Boesen et al. (1994) Biotherapy 6:291-302: Clowes et al. (1994) J. Clin. Invest. 93:644-651; Kiem et al. (1994) Blood 83:1467-1473; Salmons and Gunzberg (1993) Human Gene Therapy 4:129-141; and Grossman and Wilson (1993) Curr. Opin. in Genetics and Devel. 3:110-114.

A lentiviral vector (Science. 1996 Apr. 12;272(5259):263-7.) can also be used to deliver genes that encode the antisense drug either in vivo or to ex vivo cells. Unlike a typical retroviral vector, the lentiviral vector can be used to deliver gene to non-dividing cells.

Alternatively, liposomes may be employed to deliver the CD24 antisense and dsRNAi encoding constructs to target tissues using methods known in the art. The liposomes may be constructed to contain a targeting moiety or ligand, such as an antigen, an antibody, or a virus on their surface to facilitate delivery to the appropriate tissue. For example, liposomes prepared with ultraviolet (UV) inactivated Hemagglutinating Virus of Japan (HVJ) may be used to deliver DNA to selected tissues (Morishita, et al.). The liposomes may also be surface-coated with phospholipid-polyethyleneglycol conjugates, to extend blood circulation time and allow for greater targeting via the bloodstream. Liposomes of this type are well known. A variety of liposome have been described in the art to deliver double-stranded nucleotide or naked DNA into cells, both for ex vivo cells, or for in vivo delivery.

Receptor-mediated endocytic pathways for the uptake of DNA may permit the targeted delivery of the CD24 antisense and dsRNAi encoding constructs to specific cell types in vivo. Receptor-mediated methods of polynucleotide delivery in vivo involve the generation of complexes between vectors and specific polypeptide ligands that can be recognized by receptors on the cell surface.

For general reviews of the methods of in vivo polynucleotide delivery (also referred to as gene therapy), see Goldspiel et al (1993) Clinical Pharmacy 12:488-505; Wu and Wu (1991) Biotherapy 3:87-95; Tolstoshev (1993) Ann. Rev. Pharmacol. Toxicol. 32:573-596; Mulligan, (1993) Science 260:926-932; and Morgan and Anderson (1993) Ann. Rev. Biochem. 62:191-217; May, 1993, TIBTECH 111(5):155-215. Methods commonly known in the art of recombinant DNA technology which can be used are described in Ausubel et al. (eds.) (1993) Current Protocols in Molecular Biology, John Wiley & Sons, NY; and Kriegler (1990) Gene Transfer and Expression, A Laboratory Manual, Stockton Press, NY.

Transgenic and Knock-in Mouse Models to Test the Effect of CD24 Blockers In Vivo

Since the major cell type in the CNS that expresses HSA is the brain vascular endothelial cells, transgenic vectors that give specific expression of human CD24 in both T cells and vascular endothelial cells are used to prepare the transgenic mice. In one preferred embodiment T cell-specific expression is achieved using a transgenic vector comprised of human CD24 open reading frame and the proximal lck promoter and vascular endothelial cell specific expression is achieved using a transgenic vector comprised of human CD24 open-reading frame and the Tie II promoter, as described in Proc. Natl. Acad Sci USA, 94:3058-63(1997). To avoid interference by the endogenous HSA, the transgenic vector is injected into the fertilized embryos from mice with a targeted mutation of mouse CD24 as described in J. Exp. Med. 185: 251-262, 1997. Alternatively, the transgenic mice expressing the CD24 gene can be bred to the CD24 (−/−) mice to avoid expression of endogenous CD24. The tissue specificity of the transgene expression is verified with anti-CD24 mAb, which is available from Pharmingen (San Diego, Calif.), by flow cytometry and immunhistochemistry according to established procedure.

Alternatively, human CD24 knock-in mice can be developed to screen for therapeutic agents targeted at the human CD24 antigen. The major advantage of the knock-in mice is that all the cells that express murine CD24 can be rendered to express human CD24 genes, and as such, the knock-in mice are more relevant for testing the efficacy and safety of drugs targeted at the human CD24 antigens. Since mouse and human CD24 protein have identical amino acid sequence in the signal peptide region (encoded) regions, the CD24 knock-in mouse can be made by replacing only the coding region in exon 2. Moreover, since a major portion of the coding region encodes for GPI-cleavage signal peptide that will be removed from the mature protein, replacement of the mouse HSA/CD24 with human CD24 can be achieved simply by replacing the 81 bp region that encode for the core region of mature mouse CD24 protein with a 96 bp region that encodes for the core region of mature human CD24 protein. Replacement of some human CD24 protein amino acids with murine counterpart may be tolerated if such replacement does not change the binding activity of these molecules to anti-human CD24 antibodies, and functional ligands of human CD24 protein. For convenience of cloning, the replacement can be significantly larger than proposed region. Once the construct is produced, it is used to transfect mouse embryonic stem (ES) cells, to select for transfectants in which at least one allele of the CD24 gene is replaced the construct through homologous recombination. The recombinant alleles can be screened by PCR and/or Southern blot according the established procedures. The recombinant ES cells are tested for functionality of the recombinant allele. Once this is verified, the ES cells are used to produce chimera mice. Further breeding yield mice that are homozygous for the knock-in alleles, which express human CD24 gene on cells that had been programmed to express CD24.

The transgenic and knock-in mice produced as described above are used to screen drugs targeted at the human CD24 molecules. One example is to screen for drugs which inhibit or ameliorate autoimmune conditions such as multiple sclerosis and diabetes. The most suitable murine model for multiple sclerosis is EAE, which is induced by immunizing mice with MOG according to a established procedure.

The preferred method for testing drugs targeted at CD24 for diabetes is to breed the human CD24 transgenic mice with non-obese diabetic (NOD) mice to cross the transgene to NOD background. The drugs targeted at HSA or its homologue are administrated at approximately 2-3 weeks to determine their ID50 in the reduction of insulitis and spontaneous diabetes.

Blocking Binding of Autoreactive T cells to Endotheial Cells In Vitro or In Vivo

The present invention also relates to a method of blocking binding of autoreactive T cells to endothelial cells, in vitro or in vivo. In one aspect, the method comprises contacting the endothelial cells with a sufficient amount of HSA or a fusion protein comprising HSA to inhibit interaction of the autoreactive T cells with HSA molecules present on the surface of the endothelial cells. The cells may be in vitro, i.e., in tissue culture, or in vivo, i.e., in the body of a mammal. Blocking interaction between the endothelial and T cells in vitro is achieved by adding the protein to a chamber that contains both T cells and endothelial cells. The amount of T cells bound to a monolayer of endothelial cells in the presence or absence of HSA protein is quantified either by counting the number of cells attached, or by other methods to quantify the number of T cells that were labeled prior to adding to the monolayer.

Interaction between the endothelial and autoreactive T cells in vivo is inhibited by injecting the protein intravenously. To quantify the extent of inhibition fluorescent labeled T cells are administered to an animal and the rolling of T cells along the blood vessel is measured using established procedures known in the art.

In another aspect, interaction of autoreactive T cells with vascular endothelial cells is blocked or reduced by inhibiting or reducing expression of the CD24 polypeptide in the autoreactive T cells, the endothelial cells or both. Expression of the CD 24 polypeptide in the target cells is accomplished by introducing CD24 antisense oligonucleotides or CD24 dsRNAi into the target cells, or alternatively, transfecting these cells with a polynucleotide which encodes the CD24 antisense oligonucleotide or CD24 dsRNAi. As used herein, transfect, refers to introduction of a polynucleotide into the cell where the polynucleotide may be incorporated into the genome of the cell, converted into an autonomous replicon, or transiently expressed. The transfection can be in vivo or ex vivo. “Ex vivo transfection” means that transfection occurs outside the body of the subject from whom the target cells were obtained. “In vivo transfection” means transfection of the target cells within the body of the subject.

All references cited herein are specifically incorporated herein in their entirety.

### EXAMPLES

The following examples are for illustration only and are not intended to limit the scope of the invention.

### Example 1

### Treatment of Animals with Experimental Autoimmune Encephalomyelitis with HSAIg

### Methods

Mice Wild type C57BL/6 mice (WT) were purchased from the National Cancer Institute (Bethesda, Md.). Mice homozygous for the disrupted HSA (produced with ES cells from C57BL/6 mice) (18) (24) or CD28 (25) (backcrossed to C57BL16 for more than 8 generations) locus have been described before and are maintained at the animal facilities of the Ohio State University Medical Center. HSA transgenic mice (HSATG) have been described previously (See Zhou, Q., Wu, Y., Nielsen, P. J., and Liu, Y. 1997. Homotypic interaction of the heat-stable antigen is not responsible for its co-stimulatory activity for T cell clonal expansion. Eur J. Immunol. 27: 2524-2528, which is specifically incorporated herein by reference.) and have been backcrossed to C57BL/6j background for more than 5 generations. Mice with HSA exclusively expressed on the T cell lineage (HSATG/HSA(−/−)) were generated by crossing HSATG with the HSA(−/−) mice.

Induction and clinical evaluation of EAE The immunogen, MOG peptide 35-55 of rat origin (MEVGWYRSPFSRVVHLYRNGK), was synthesized by Research Genetics, Inc. (Huntsville, Ala., USA). The purity of the peptide was >90%. Mice of 8-12 wks of age were immunized subcutaneously with 200 μg MOG peptide in complete Freund's Adjuvant (400 μg of Mycobacterium tuberculosis per ml) in a total volume of 100 μl. They received 200 μg of Pertusis toxin (List Biological, Campbell, Calif.) in 200 μl PBS in the tail vein immediately after the immunization, and again 48 hours later. The mice were observed every other day and scored on a scale of 0-5 with gradations of 0.5 for intermediate scores: 0, no clinical signs; 1, loss of tail tone; 2, wobbly gait; 3, hind limb paralysis; 4, hind and fore limb paralysis; 5, death. T cell proliferation assay Draining lymph node cells were isolated 10 days after immunization. 5×10⁵ cells/well were stimulated with given concentrations of MOG peptide in the presence 6×10⁵ cells/well of irradiated (2,000 rad) syngeneic splenocytes for 60 hours. The cultures were pulsed with ³H-thymidine (1 μCi/well; ICN Pharmaceuticals Inc., Costa Mesa, Calif. USA) for another 12 hours, and incorporation of 3H-thymidine was measured in a liquid scintillation P-plate counter.

ELISpot assay to evaluate frequencies of T cells that produce IFN-γ, IL-2 and IL-4 upon restimulation with MOG peptide in vitro The antibody pairs and the procedures have been described (20), except that the MOG peptide was used for stimulation at 10 μg/ml. The numbers presented are those of cytokine producers per million of draining lymph node cells.

Histology

Mice were sacrificed by CO₂ inhalation. Spinal cords were removed by insufflation and fixed in 10% formalin/PBS. Paraffin sections were prepared and stained with hematoxylin and eosin. Neurological lesions were graded on each of the 10 cross sections per spinal cord, according the following criteria: 0, no infiltrate; 1, 3 or less focal meningeal infiltrates; 2, more than 3 focal meningeal infiltrates; 3, up to 5 perivascular infiltrate foci in the parenchyma with involvement of less than 5% of the white matter; 4, 5-10 perivascular foci in the parenchyma or invasions involving 5-25% the white matter; 5, more than 10 perivascular foci or diffuse infiltration involving more than 25% of the white matter.

Passive Transfer of EAE

Groups of 8-10 WT and HSA(−/−) mice were immunized with 200 μg of MOG peptide subcutaneously. At 10 days after immunization, draining lymph nodes were harvested and stimulated at 4×10⁶/ml in Click's EHAA medium supplemented. with 15% fetal calf sera, 5% IL-2 supernatant, and 50 μg/ml of MOG peptide for 4 days. 1×10⁸ cells were injected i.p. into each recipient mouse that had been γ-irradiated (550 rad) 1 h earlier.

Preparation of Fusion Protein and Treatment of EAE z

The HSA fragment encoding the signal peptide and the mature protein sequence were amplified by PCR, using GGA AAG CTT ATG GGC AGA GC, SEQ ID NO.:6, as forward primer, CGA GAT CTC TGG TGG TAG CG, SEQ ID NO.:7, as reverse primer, and HSA cDNA as template. The PCR products were digested with Hind III and Bgi II enzymes and were ligated to Hind III and Xba I-digested pCDM8 vector (Invitrogen, San Diego) and a Mba I and Bam HI-treated DNA fragment encoding human IgG1 Fc, which were amplified by PCR using CAG GGA TCC CGA GGG TGA GTA CTA AGC TAG CTT CAG CGC TCC TGC CTG, SEQ ID NO.:7, as forward primer and CTT CGA CCA GTC TAG AAG CAT CCT CGT GCG ACC GCG AGA GC, SEQ ID NO.:8, as reverse primer, and DNA from human peripheral blood as template. The construct was verified by DNA sequencing and was used to transfect the Chinese Hamster Ovary cell line. The cells that secreted HSAIg fusion protein were amplified in DMEM containing 5% fetal calf serum until confluence. The cell monolayers were washed with serum-free medium and cultured in optimal M medium for 72 hours. The supernatants were collected and the HSAIg was purified using a protein G column according to the manufacturer's protocol. The purity of the protein was verified by SDS PAGE.

### Results

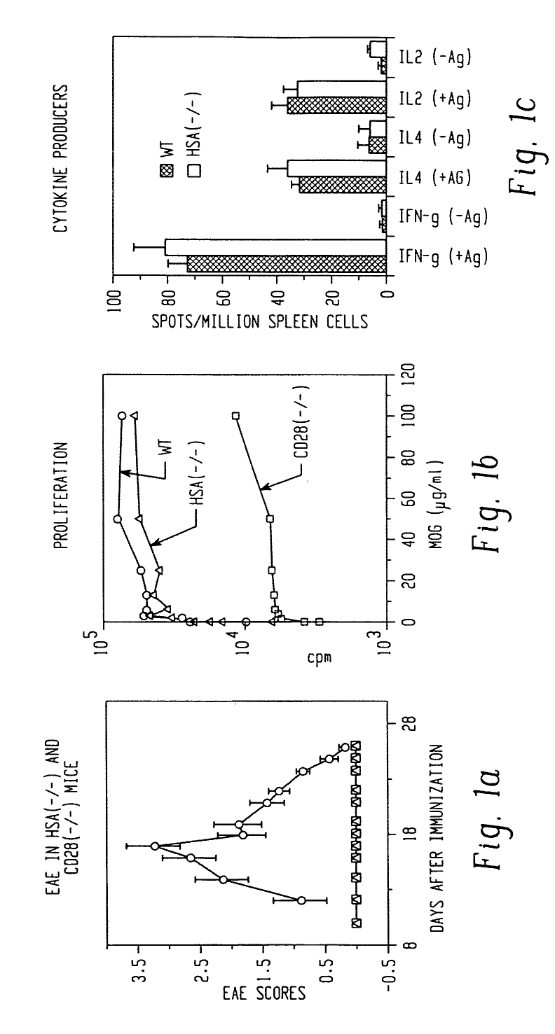

To test if HSA is essential for the development of EAE, we immunized C57BL/6 wild-type (WT), and HSA- or CD28-deficient mice with myelin oligodendrocyte glycoprotein (MOG) peptide AA35-55 in conjunction with complete Freund's adjuvant and pertusis toxin. As shown in FIG. 1a, wild-type mice developed acute EAE within two weeks of peptide immunization, while those with targeted mutation of either HSA or CD28 were completely resistant to EAE induction. Interestingly, while targeted mutation of CD28 ablated induction of MOG-specific T cells, as revealed by proliferative response of draining lymph node cells, that of HSA had little effect on peptide-specific T cell proliferation (FIG. 1b). Moreover, the frequencies of antigen-specific, IL2-, IL4-, and IFNγ-producing cells were not altered in HSA(−/−) mice (FIG. 1c). The anti-MOG peptide IgG responses were also detected in HSA-deficient mice (data not shown). The differential effects of HSA and CD28 mutations on T cell priming reveal that these genes mediate two distinct checkpoints in the development of EAE: CD28 controls induction of auto-reactive T cells, while HSA determines their pathogenicity.

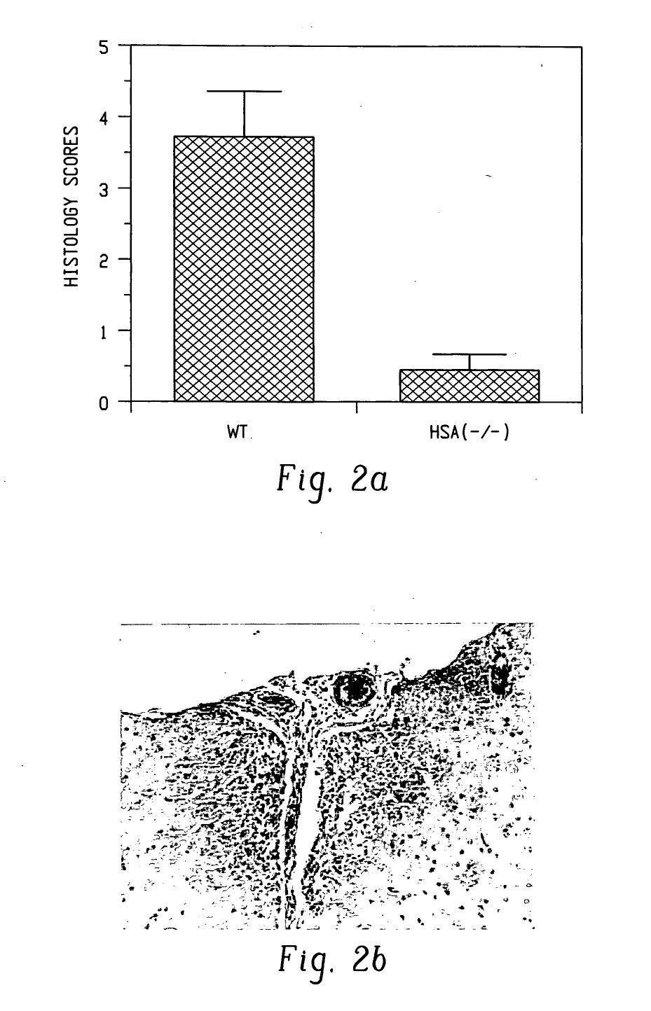

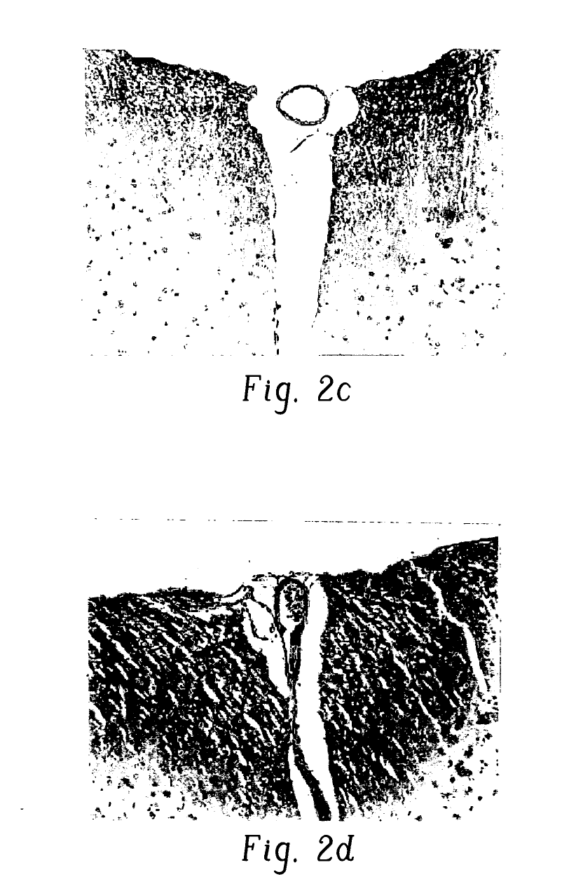

Histological analysis of MOG-peptide immunized WT and HSA-confirms the clinical scores. The histological scores were summarized in FIG. 2a, while representative histology sections were presented FIG. 2b-d. As shown in FIG. 2b, active immunization with MOG peptide induces multiple neurological lesions in the wild-type mice, characterized by multiple lesions with extensive invasion of parenchyma. In contrast, the spinal cords of HSA-KO mice are either devoid of any lesion (FIG. 2c), or with one or two low grade lesions involving meninges (FIG. 2d).

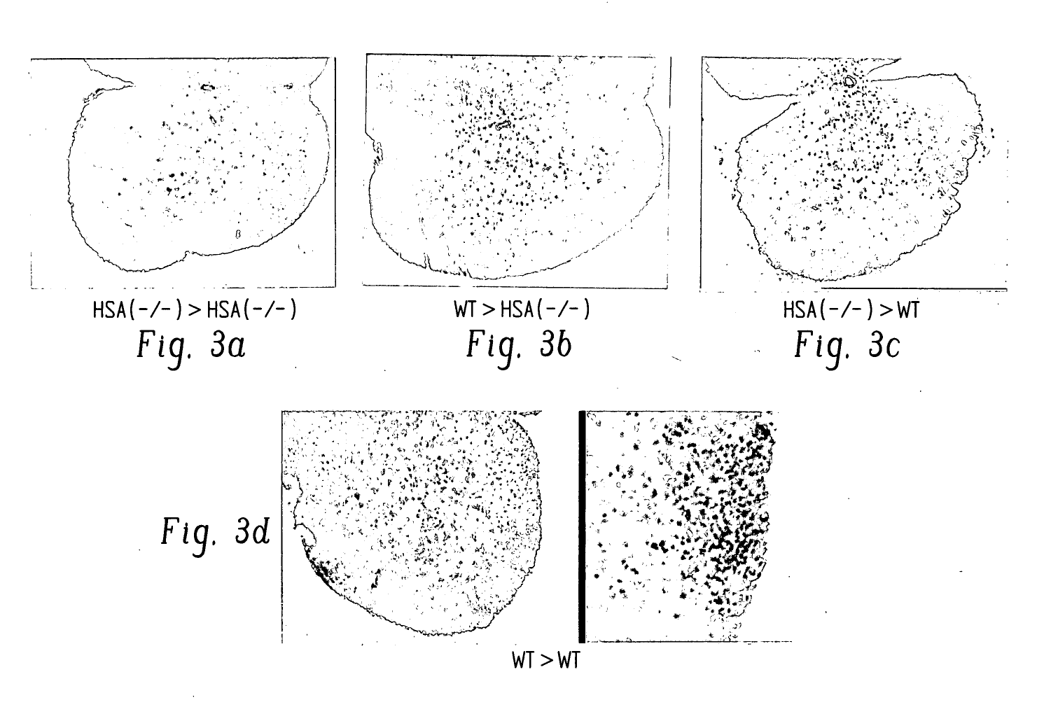

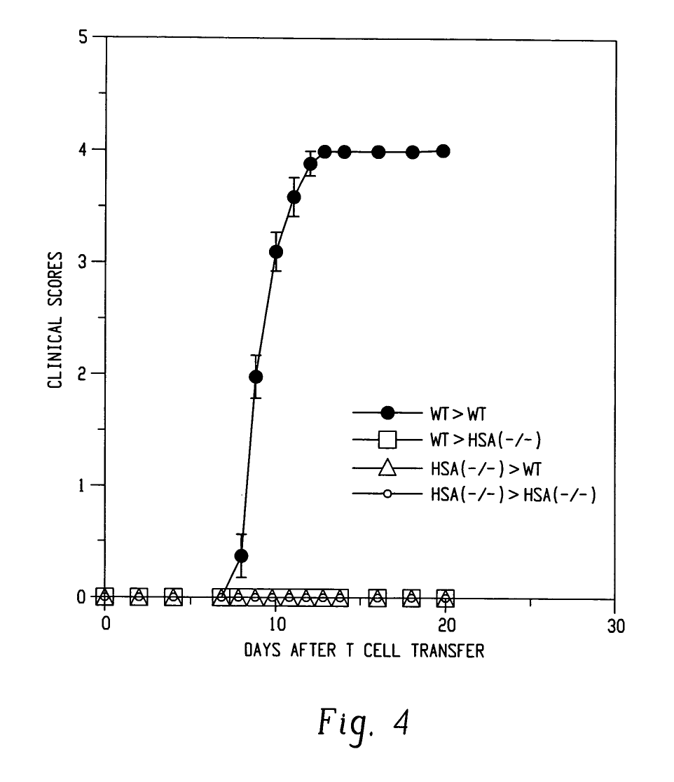

We adoptively transferred activated draining lymph node cells to WT and HSA-deficient recipients. As shown in FIGS. 3 and 4, WT T cells induced severe EAE in WT T recipients within 8 days of adoptive transfer. Interestingly, none of the HSA-deficient recipients developed EAE. Thus HSA expression on T cells alone appears insufficient for EAE development. Moreover, T cells from HSA-deficient mice failed to induce disease regardless of HSA gene status in the recipient, which indicates that HSA expression on T cells is necessary for EAE development. These results strongly suggest that HSA must be expressed on both host cells and auto-reactive T cells in order to induce EAE.

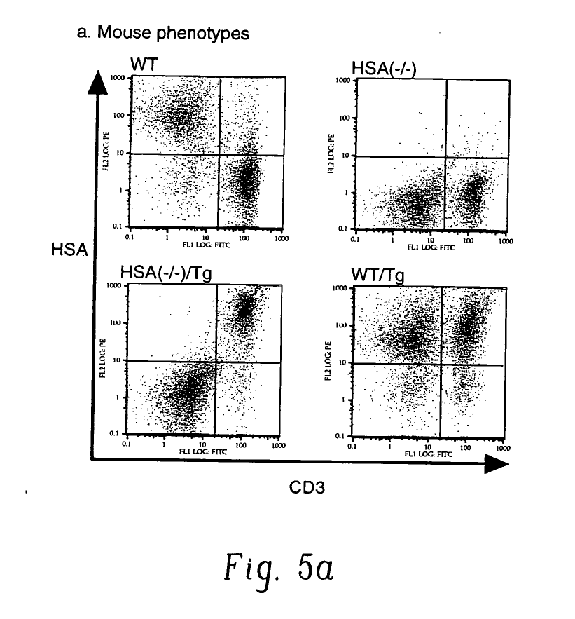

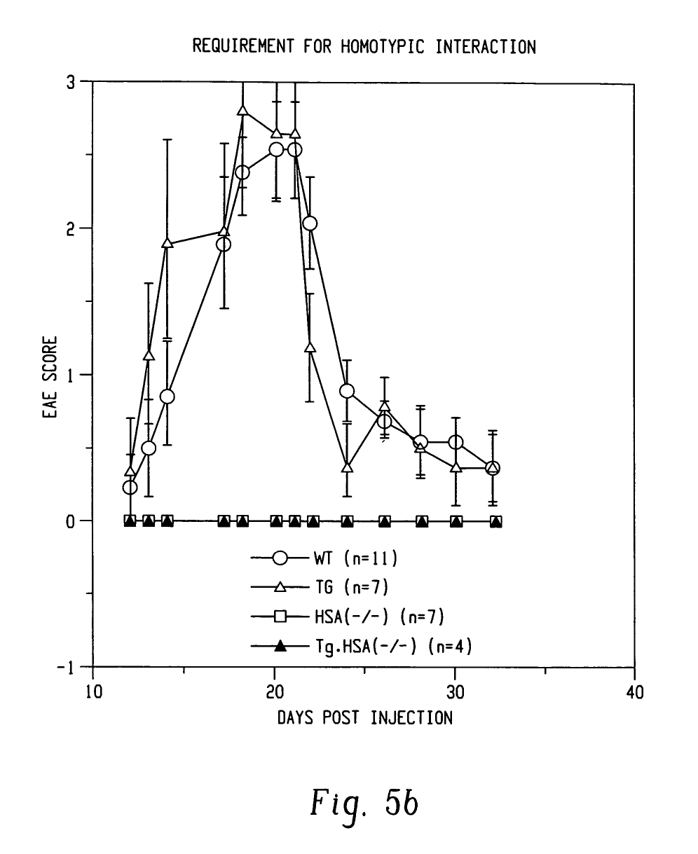

To substantiate these observations, we produced mice that expressed HSA exclusively on T cells. We have previously reported the transgenic mice in which expression of HSA was under the control of the Ick proximal promoter (HSATG) (22). For this study, We crossed the HSA transgene to HSA-deficient mice to produce mice that expressed HSA exclusively on T cells (FIG. 5a). To test if HSA expression on the T cell lineage is sufficient for EAE development, we immunized WT, HSA-TG, HSA(−/−) and HSATG HSA(−/−) mice with MOG. As shown in FIG. 5b, wild-type and HSATG mice developed EAE with essentially identical kinetics, which indicates that transgenic expression of HSA on T cells does not prevent the production and effector function of self-reactive T cells. Nevertheless, much like HSA (−/−) mice, the mice with exclusive HSA-expression on the T cell lineage failed to develop EAE. These results demonstrated clearly that HSA expression on T cell lineage alone is insufficient for EAE development.

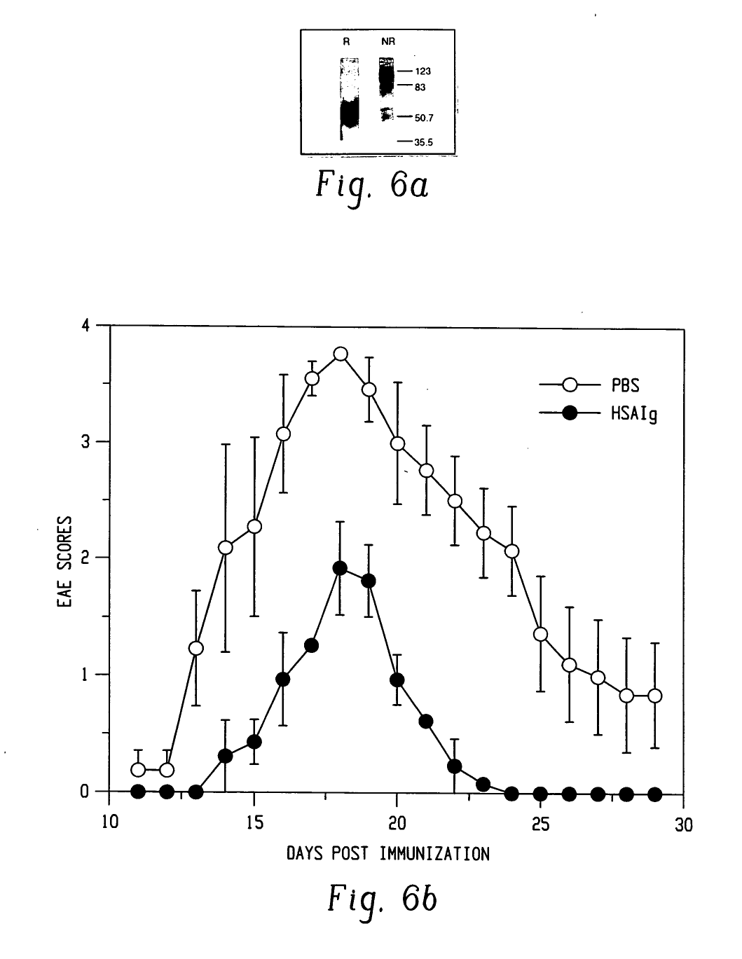

The fact that HSA may be a critical checkpoint after activation of self-reactive T cells suggests a novel approach in treating autoimmune neurological diseases. Since an anti-HSA mAb was toxic in the EAE model to address this issue (Data not shown), we produced a fusion protein between the extracellular domain of HSA and the Fc portion of human IgG1, to block the HSA-mediated interactions. As shown in FIG. 6a, the fusion protein has an apparent molecular weight of about 100 kD under non-reducing SDS-PAGE. After reduction, it migrated as a 50 kD band. We treated mice starting at 8-10 days after immunization with MOG peptide, when MOG-specific T cells response had already expanded in the local lymph nodes. As shown in FIG. 6b, HSAIg drastically ameliorated EAE. All HSA1 g-treated mice recovered substantially earlier than did the control mice. Since MOG-reactive T cells had been activated prior to HSAIg administration, the clinical signs in the treated group may reflect the fact that some autoreactive T cells had already migrated into the central nervous system.

HSAIg, a fusion protein consisting of the extracellular domain of mouse HAS and the Fc portion of immunoglobulin, drastically ameliorates the clinical sign of EAE even when administrated after self-reactive T cells had been expanded. Thus, identification of HSA as a novel checkpoint, even after activation and expansion of self-reactive T cells, provides a novel approach for immunotherapy of autoimmune neurological diseases, such as multiple sclerosis.

### Example 2

### Production Human CD24Ig Fusion Protein

Fragments of the human CD24 polypeptides lacking the GPI anchor region are fused with human Ig constant region to form CD24-Ig fusion protein. In one embodiment the CD24 polypeptide fragment comprises the signal peptide. In another embodiment the CD24 polypeptide fragment lacks the signal peptide. The fragment of the human CD24 coding sequence is subcloned into vector pIg (from Novagen) Hind III and BamHI sites. Suitable primers useful in subcloning include, but are not limited to, CD24 forward primer (CD24F.H3): G GCC AAG CTT ATG GGC AGA GCA ATG GTG, SEQ ID NO.:9, with Hind III site 5′ to ATG start codon. CD24-Ig reverse primer (CD24Rig.Bm): GG CCG GAT CCA CTT ACC TGT CGC CTT GGT GGT GGC ATT, SEQ ID NO.10, with Bam HI site and the SD sequence (A CTT ACC TGT, SEQ ID NO.:11) next to 3′ end of TTKA (direct sequence: ACC ACC AAG GCG, SEQ ID NO.:12) in Human CD24. The construct is transfected into CHO cells, and the CD24Ig is secreted into the tissue culture medium. CD24Ig is purified by affinity chromatography using a Protein G column. The clone compresses CD24 signal peptide, CD24 core peptide and the IgG/Fc portion, but lacks the GPI anchor signaling region.

### Example 3

### Production of Anti-Human CD24 mAb that Blocks Autoreactive T Cells-Initiated Tissue Destruction

Human CD24 coding sequence is subcloned into vector pCDM8 (from Invitrogen) Hind III and Xho I sites. CD24 forward primer (CD24F.H3): G GCC AAG CTT ATG GGC AGA GCA ATG GTG with Hind III site 5′ to ATG start codon. CD24 reverse primer (CD24R. Xho): A TCC CTC GAG TTA AGA GTA GAG ATG CAG with Xho I site 3′ to TAA stop codon. The CD24 cDNA is transfected into murine 3T3 cells. The 3T3 cell lines that stably express human CD24 molecules are used to immunize syngeneic mice. After 2-3 immunization, spleen cells are fused with myeloma AgX865, after selection with HAT medium the supernatants are screened for anti-human CD24 mAbs. The antibodies are tested for their ability to block both adhesion of human T cells to human endothelial cells in vitro, and their ability to block human CD24-mediated T cell trafficking to target tissues, such as the pancreas and the central nervous system using the transgenic model detailed below.

### Example 4

### Testing Putative Inhibitors of Multiple Sclerosis with CD24 Transgenic and Knock-in Mice

The immunogen, MOG peptide 35-55 of rat origin (MEVGWYRSPFSRVVHLYRNGK, SEQ ID NO.:13), is available from Research Genetics, Inc. (Huntsville, Ala., USA). Mice of 8-12 wks of age are immunized subcutaneously with 200 μg MOG peptide in complete Freund's Adjuvant (400 μg of Mycobacterium tuberculosis per ml) in a total volume of 100 μl. They receive 200 μg of Pertusis toxin (List Biological, Campbell, Calif.) in 200 Id PBS in the tail vein immediately after the immunization, and again 48 hours later. The mice are observed every other day and scored on a scale of 0-5 with gradations of 0.5 for intermediate scores: 0, no clinical signs; 1, loss of tail tone; 2, wobbly gait; 3, hind limb paralysis; 4, hind and fore limb paralysis; 5, death. The putative inhibitory molecules are injected at 1 week after immuunization. Those that substantially reduce the clinical score of EAE are selected for further testing.

### Example 5

### Generation of Human CD24 Gene Knock-in Mice

The basic strategy used to produce CD24 gene knock-in mice is to replace part of murine CD24 gene exon 2 sequence with that of human CD24 sequence. We took advantage of the fact that signal peptide, encoded by exon 1 of mouse and human CD24 gene, are identical between mouse and human CD24. We therefore replaced only part of the mouse exon 2 sequence with that of 240 bp of human CD24. The construct with the desired sequence is shown in FIG. 13.

As shown in FIG. 13, arm 1 of the construct comprised of a 2.7 kb fragment of mouse CD24 gene, cloned from 129R1 ES cells (Seq ID. 20). The arm 2 of the construct is a chimera gene consisting of the last 256 bp sequence of CD24 exon 1, first 240 bp human CD24 exon 2 sequence and about 3 kb of mouse CD25 sequence comprising of both remaining exon 2 sequence encoding for 3′ untranslated region and 3′ sequence of the CD24 gene Seq I.D. 22). The construct is used to transfect ES cells. The recombinants are screened by procedures established in the art, including PCR and Southern blot. The ES cells with the illustrated knock-in alleles are transfected with plasmid encoding Cre recombinase that recognize the lox P sequence. Since ES cells expression CD24 gene, as revealed by cell surface flow cytometry, the functionality of the knock-in alleles can be confirmed by cell surface expression of human CD24. The ES cells with the capacity to express human CD24 are used to produce chimera mice by blastocyte injection according to technique known in the art. Mice with germ-line transmission are produced by breeding the chimera mice.

### Example 6

### Inhibition of CD24 Expression by dsRNAi Technology

Mouse and human CD24 genes are highly homologous. It is therefore possible to select regions that are identical between mouse and human CD24 as target for dsRNAi drug. An alignment between Human CD24 (XM_{—}099027) and Mouse CD24 (NM_{—}009846) is shown in FIG. 14. Eight regions with a stretch of identical nucleotide that is 17 bp or longer are highlighted and as preferred target sequences. Although identity between mouse and human is not an essential feature of the dsRNAi molecule, targeting the dsRNAi to identical regions provides a dsRNAi which can be used to inhibit expression of both mouse and human CD24 genes. As a result, preclinical small rodent models can be used to screen for the efficacy of dsRNAi molecule in animal disease models, in addition to cell culture.

CHO cells transfected with either mouse or human CD24 cDNA are transfected with dsRNAi, produced by in vitro annealing. Briefly, both sense and antisense RNA corresponding to nt. 46-64 (+1 as translation starting site) of mouse and human CD24 gene plus two thymidine were synthesized by a commercial vendors. The sequence of the two strands are as follows: CD24-46/64 iRNA.F: 5′-CUG GCA CUG CUC CUA CCC ATT-3′ (seq ID. 16), and CD24-46/64 iRNA.R: 5′-UGG GUA GGA GCA GUG CCA GTT-3′ (seq ID 17). Control oligonucleotides were designed based the inverted sequence, as follows invCD24-46/64 iRNA.F: 5‘-ACC CAU CCU CGU CAC GGU C TT-’ (seq ID. 18) invCD24-46/64 iRNA.R: 5′-GAC CGU GAC GAG GAU GGG UTT-3′ (seq ID 19). For annealing of siRNA, 20 uM single strands will be incubated in annealing buffer (100 mM KOAc, 30 mM HEPES at pH7.4, 2 mM MgAc) for 1 min at 94 degree followed by 1 h at 37 degree and resulting dsRNA. The resulting dsRNAi is used to transfect CHO cells. At 48 hours after transfection, the cells are analyzed for CD24 expression by flow cytometry.

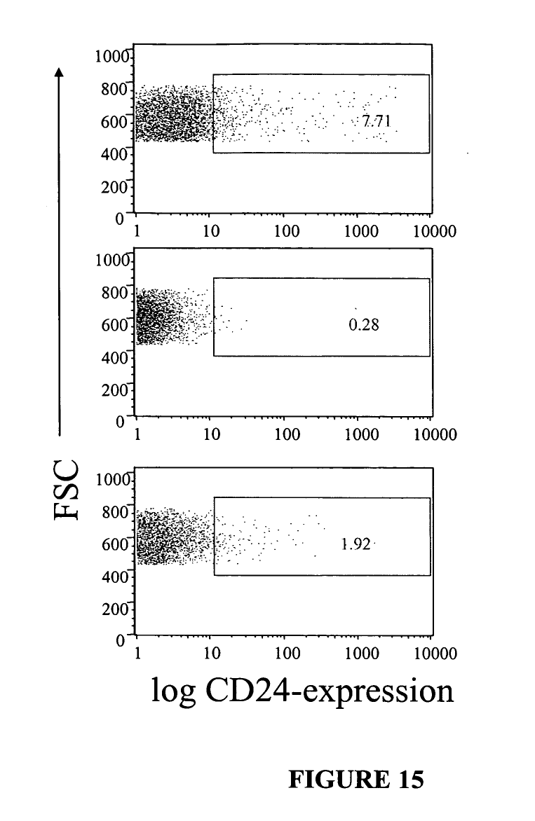

As shown in FIG. 15, transient transfection lead to expression of CD24 on about 7% of the CHO cells. Inverted dsRNA reduced expression of CD24 some what, although significant number of CHO cells (2%) still express high level of CD24. Importantly, the expression of CD24 is completely abrogated when the CHO cells are co-transfected with dsRNAi corresponding to human/mouse CD24 sequence. These results revealed that the dsRNAi can be used to inhibit expression of CD24.

1. A method for inhibiting autoreactive T cell-initiated destruction of tissues in a mammal comprising:
administering a pharmaceutical composition to the mammal, said pharmaceutical composition comprising an agent selected from a fusion protein comprising an HSA/CD24 polypeptide, and a fusion protein comprising a biologically active fragment of an HSA/CD24 polypeptide, and an anti HSA/CD24 antibody; wherein said pharmaceutical composition is administered in an amount sufficient to reduce autoreactive T cell-initiated tissue destruction in said mammal.

2. The method of claim 1, wherein the HSA/CD24 polypeptide or fragment thereof is glycosylated.

3. A method for treating a patient suspected of having, known to have, or predisposed to having an autoimmune disease, said method comprising.
administering to said patient a pharmaceutical composition comprising an agent selected from a fusion protein comprising an HSA/CD24 polypeptide; fusion protein comprising a biologically active fragment of an HSA/CD24 polypeptide.

4. The method of claim 3, wherein the HSA/CD24 polypeptide or fragment thereof is glycosylated.

5. (canceled)

6. (canceled)

7. The method of claim 1, wherein the agent is a fusion protein comprising a peptide tag linked by a peptide bond to a core region of the HSA/CD24 polypeptide.

8. The method of claim 7, wherein the HSA/CD24 polypeptide or fragment thereof is glycosylated.

9. The method of claim 8, wherein the peptide tag is human IgG 1 Fc and the polypeptide is the human CD24 polypeptide.

10. (canceled)

11. (canceled)

12. (canceled)

13. (canceled)

14. (canceled)

15. (canceled)

16. A method of blocking binding of autoreactive T cells to vascular endothelial cells, comprising:
contacting said vascular endothelial cells with an agent selected from a fusion protein comprising an HSA/CD24 polypeptide- and a fusion protein comprising a biologically fragment of an HSA/CD24 polypeptide, wherein said cells are contacted with a sufficient amount of said agent to inhibit interaction of the autoreactive T cells with the vascular endothelial cells.

17. The method of claim 16, wherein the HSA/CD24 polypeptide or fragment thereof is glycosylated.

18. A method of reducing autoreactive T cell-initiated destruction of tissues in a mammal, comprising: administering to the mammal an agent that reduces interaction of the CD24 polypeptide that is present on the autoreactive T cells with a functional ligand of the CD24 polypeptide wherein the agent is chosen from a fusion protein comprising an HSA/CD24 polypeptide and a fusion protein comprising a biologically active fragment of an HSA/CD24 polypeptide.

19. (canceled)

20. (canceled)

21. (canceled)

22. (canceled)

23. (canceled)

24. (canceled)

25. (canceled)

26. (canceled)

27. (canceled)

28. A method for inhibiting autoreactive T cell-initiated destruction in a mammal by targeting CD24 molecules at genomic, transcription, post-transcriptional, translational or ligand binding levels, the method comprising:
administering a pharmaceutical composition to the mammal, said pharmaceutical composition comprising an agent selected from a fusion protein comprising an HSA/CD24 polypeptide-, and a fusion protein comprising a biologically active fragment of an HSA/CD24 polypeptide;
wherein said pharmaceutical composition is administered in an amount sufficient to reduce autoreactive T cell-initiated tissue destruction in said mammal.


SEQUENCE LISTING (16 sequences)

SEQ ID NO: 1 - protein, 76 aa, Mus musculus
MGRAMVARLGLGLLLLALLLPTQIYCNQTSVAPFPGNQNISASPNPSNATTRGGGSSLQSTAGLLALSLSLLHLYC

SEQ ID NO: 2 - protein, 80 aa, Homo sapiens [sequence omitted]

SEQ ID NO: 3 - protein, 76 aa, Rattus norvegicus
MGRAMVVRLGLGLLLLALLLPTQIYCNQTSVAPFSGNQSISAAPNPTNATTRSGCSSLQSTAGLLALSLSLLHLYC

SEQ ID NO: 4 - DNA, 1494 bp, Artificial
  gene (1)..(157): mouse HSA
atgggcagagcgatgggggccaggctagggctggggttgctgcttctggcactgctcctacccacgcagatttactgcaaccaaacatctgttgcaccgtttcccggtaaccagaatatttctgcttccccaaatccaagtaacgctaccaccagagatcccgagggtgagtactaagctagcttcagcgctcctgcctggacgcatcccggctatgcagccccagtccagggcagcaaggcaggccccgtctgcctcttcacccggagcctctgcccgccccactcatgctcagggagagggtcttctggctttttcccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagccggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtcctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagccccgctccccgggctctcgcggtcgcacgaggatgctt

SEQ ID NO: 5 - DNA, 864 bp, Artificial
  gene (1)..(156): mouse HSA
atgggcagagcgatgggggccaggctagggctggggttgctgcttctggcactgctcctacccacgcagatttactgcaaccaaacatctgttgcaccgtttcccggtaaccagaatatttctgcttccccaaatccaagtaacgctaccaccagagatcccgaggagcccaaatcttgtgacaaaactcacacatgcccaccgtgcccaggcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - DNA, 20 bp, Mus musculus
ggaaagcttatgggcagagc

SEQ ID NO: 7 - DNA, 20 bp, Mus musculus
cgagatctctggtggtagcg

SEQ ID NO: 8 - DNA, 41 bp, Homo sapiens
cttcgaccagtctagaagcatcctcgtgcgaccgcgagagc

SEQ ID NO: 9 - DNA, 28 bp, Artificial
  primer_bind (1)..(10): Hind III restriction endonuclease recognition site
ggccaagcttatgggcagagcaatggtg

SEQ ID NO: 10 - DNA, 38 bp, Artificial
  primer_bind (1)..(10): BamH I restriction endonuclease recognition site
ggccggatccacttacctgtcgccttggtggtggcatt

SEQ ID NO: 11 - DNA, 10 bp, Homo sapiens
acttacctgt

SEQ ID NO: 12 - DNA, 12 bp, Homalozoon vermiculare
accaccaaggcg

SEQ ID NO: 13 - protein, 21 aa, Rattus norvegicus
MEVGWYRSPFSRVVHLYRNGK

SEQ ID NO: 14 - protein, 288 aa, Artificial
  DOMAIN (1)..(52): mouse HSA
MGRAMGARLGLGLLLLALLLPTQIYCNQTSVAPFPGNQNISASPNPSNATTRDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKZ

SEQ ID NO: 15 - DNA, 48 bp, Homo sapiens
cagggatcccgagggtgagtactaagctagcttcagcgctcctgcctg

SEQ ID NO: 16 - DNA, 28 bp, Artificial
  primer_bind (1)..(14): human CD24
atccctcgagttaagagtagagatgcag